(19) United States
(12) Patent Application Publication
(10) Pub. No.: US 2021/0137813 A1
(43) Pub. Date: May 13, 2021

(54) COSMETIC COMPOSITION COMPRISING A CAESALPINIA SPINOSA EXTRACT, A KAPPAPHYCUS ALVAREZII EXTRACT, AT LEAST ONE PREBIOTIC AND A PROBIOTIC

(71) Applicant: L V M H RECHERCHE, SAINT JEAN DE BRAYE (FR)

(72) Inventors: Marc DUMAS, SAINT JEAN LE BLANC (FR); David NOTTE, ORLEANS (FR); Françoise PELLICIER, LOURY (FR)

(73) Assignee: L V M H RECHERCHE, SAINT JEAN DE BRAYE (FR)

(21) Appl. No.: 16/956,047

(22) PCT Filed: Dec. 21, 2018
(86) PCT No.: PCT/FR2018/053502

§ 371 Date: Dec. 3, 2020

(30) Foreign Application Priority Data
Dec. 22, 2017 (FR) 1763139

(51) Int. Cl. A61K 8/73; A61K 8/9722; A61K 8/99; A61Q 1/04; A61Q 1/10; A61Q 19/00

(52) U.S. Cl. CPC A61K 8/737; A61K 8/9722; A61K 8/99; A61K 2800/591; A61Q 1/10; A61Q 19/00; A61K 2800/85; A61Q 1/04

(57) ABSTRACT

The present invention relates to a cosmetic composition comprising, in a physiologically-acceptable medium at least: a) a cosmetic agent consisting of galactomannans of molar masses comprised between 5 and 630 kDa, preferably between 5 and 120 kDa, still more preferably between 8 and 80 kDa, and crosslinked sulfated galactans of molar masses comprised between 7 and 3000 kDa, preferably between 7 and 1100 kDa, still more preferably between 8 and 200 kDa, and b) a prebiotic and a probiotic.

### FIELD OF THE INVENTION

The present invention relates to a cosmetic composition comprising a cosmetic agent consisting of galactomannans and cross-linked sulfated galactans and at least one prebiotic and a probiotic, and its use, notably to maintain the balance of the skin microbiota and promote or improve the wear of makeup.

### STATE OF THE ART

The skin, the largest organ in the human body, is colonized by billions of microorganisms (bacteria, yeast, fungi, viruses, etc.) collectively called “microbiota” or “microflora”. The term “skin microbiome” designates all these microorganisms, their genome and their interactions with their environment.

The number of bacteria present on the skin can reach millions per cm².

The human skin flora can be subdivided into two groups:
Transient flora composed of fungi, viruses and bacteria, harmless for the most part, called saprophytes, which feed on decomposing organic matter originating from the environment. This flora is not permanent, it varies over the day, depending on the activities performed and variations in environmental conditions and the exposure of individuals to these conditions. The most common transient species are Staphylococcus aureus, Escherichia coli, Pseudomonas aeruginosa and Bacillus species;
The resident flora is composed of commensal microorganisms, i.e., living on their host without causing it damage. The composition of these flora is fixed, and after disturbance, its same components reform spontaneously. These microorganisms (Gram positive and negative bacteria, etc.) live on the epidermis and are mainly found in the upper layers of the stratum corneum and in the ducts of the sweat glands and pilosebaceous follicles and the epidermal appendages like the nails and the hair.

These microorganisms are indispensable to life. In addition to their role in the appearance of body odor, they are closely linked to maintaining healthy skin. Resident and transient microbes do not cause illness or dysfunction under normal conditions, i.e., when adequate hygiene is ensured and when the resident flora, the immune responses and the barrier function of the skin are intact. The skin microbiota is therefore capable of playing a barrier role and protecting its host.

At the same time, the skin generates antimicrobial lipids and peptides such as β-defensins, receptors dedicated to the recognition of pathogens which, all together, form “skin innate immunity”.

By growing and replicating, resident bacteria also produce toxic metabolites, bacteriocins, and serine proteases such as those of Staphylococcus epidermidis to inhibit the growth of other microorganisms and prevent the formation of their biofilm, by which they adhere to the skin. Commensal flora stimulate the skin barrier and innate immunity, thereby maintaining healthy and strong skin. The balance of the skin microbiota as well as the expression of the environmental conditions of the skin (temperature, pH, salinity, hormone contents, lipid, protein, water and oxygen contents, UV exposure) are also essential to this maintenance.

In addition to intrinsic factors such as age, sex, genetic make-up and immune reactivity, extrinsic factors such as climate (ambient temperature and humidity, UV) drug treatments, the use of cosmetics for hygiene, care or make-up can have a significant impact on the composition of skin microbial communities.

It has also been demonstrated that the composition of the skin microbiota is influenced by the ability of the skin to retain the water that it contains and the production of sebum. The sebum level is negatively correlated with 3 diversity indices from 16S taxonomic data (order, family, genus and species) and the same trend has been observed for an insensible water loss (reflecting the quality of the skin barrier).

These studies presented at the ASCB Meeting in San Francisco in December 2016 by C Heusèle et al. and entitled “Microbiota diversity on healthy women's face in relation to skin biophysical characteristics”, show a correlation between sebum production and the barrier function of the skin with the composition and diversity of the microbiota.

The variability of the human cutaneous microbiota can then lead to alteration in its structure (dysbiosis) and have health consequences by causing skin dysfunctions or contributing to the development of diseases via the colonization and proliferation of the resident and/or transient flora. For example, a reduction in microbial diversity is observed in certain pathologies such as atopic dermatitis (Flores G et al. J Drugs Dermatol 2014: 13(11): 1365-72) and psoriasis (Alekseyenko A et al., Microbiome 2013: 1(131): 2-17)

The application of makeup compositions, in particular, such as foundation, can thus disturb the balance of the skin microbiota, in particular by disturbong bacterial diversity. The invisible film of makeup applied onto the skin can also temporarily cause a change in the water-lipid balance of the skin and desquamation, factors that are likely to deregulate the balance of the skin microbiota.

The invisible film of makeup applied onto the skin may also temporarily cause a condition of hypoxia likely to change the skin microbiota, in particular the aerobic organisms that live on the surface of integuments where the oxygen content is the highest. In fact, it has been demonstrated that an application of foundation significantly impacts bacterial diversity (Staudinger T et al. J Appl. Microbiol 2011: 110 (6): 1381-9.

The lipid film constituted by an excessive sebum production can also temporarily cause a condition of hypoxia likely to change the skin microbiota, in particular of the aerobic organisms that live on the surface of integuments where the oxygen content is the highest.

But hypoxia is also capable of altering the skin tissue itself at several levels:
by reducing the formation of desmoglein-1, a constituent protein of the cohesive bonds among the cells and therefore the strength of the epidermis (Straseski J et al. Wound Repair Regen. 2009; 17(4): 606-616),
also by reducing the formation of aquaporin 3, a protein that enables water to be brought into the epidermis to hydrate the skin (Straseski J et al. Wound Repair Regen. 2009; 17(4): 606-616),
by stimulating the production of reactive oxygen species (ROS) that alter skin constituents and cells (Nys K et al. Free Radic Biol Med. 2012;52(6):1111-20)
by inhibiting the formation of keratins for differentiation such as keratin 10 (Straseski J et al. Wound Repair Regen. 2009; 17(4): 606-616), and/or
by increasing the presence of matrix metalloproteinases (MMP) (Xia YP et al. J. Invest.

Dermatol. 2001; 116: 50-56) that degrade the constituents of the skin such as collagen, which may impact skin density and firmness and consequently pore opening and the formation of fine lines and wrinkles.

Therefore, the interest in makeup compositions for keratin materials, especially the skin, or compositions for oily skin or skin with an oily tendency is understood, to have ingredients able to preserve the skin ecosystem from the consequences of hypoxia by ensuring at least one of the following protective actions:
preserving the microbiota and its diversity by providing it with the skin conditions that allow this, such as strengthening the skin barrier and controlling sebum production,
reinforcing the cohesion of the epidermis to strengthen the skin,
stimulating the formation of the skin barrier to maintain sufficient hydration in the skin,
detoxifying the skin of reactive oxygen species,
inhibiting the formation of MMPs, and/or
stimulating the formation of hypoxia-inducible factor (H IF) which protects skin cells from hypoxia.

Therefore there is a need to develop new cosmetic makeup compositions for keratin materials, in particular for the skin, capable of preserving the diversity of the microbiota, controlling its growth and improving the quality of the keratin materials (skin, mucosa, integuments) and especially the skin, the relief and texture of its surface that constitutes an ecological niche for these microorganisms, in particular to maintain the skin ecosystem and/or maintain the balance of the skin microbiota, in particular microbial diversity, reinforce the skin barrier, improve the resistance of the skin to stress, in particular, prevent and/or limit the consequences on the skin of a hypoxic environment, reduce skin inflammation, promote desquamation and/or epidermal renewal, reduce the loss of firmness involved in decreased elasticity of the pores and the formation of wrinkles and fine lines, improve the uniformity of the complexion, and/or promote and/or improve makeup wear and the uniformity of its distribution on the surface of the skin.

The Applicant has demonstrated that the combination of a cosmetic agent consisting of galactomannans and crosslinked sulfated galactans, a prebiotic and a probiotic, meets this need. In particular, the Applicant was able to show that a composition comprising a cosmetic agent consisting of galactomannans obtained from Caesalpinia spinosa of molar masses comprised between 1 and 150 kDa and crosslinked sulfated galactans obtained from Kappaphycus alvarezii of molar masses comprised between 1 and 150 kDa, and a pre/probiotic complex comprising an alpha-glucooligosaccharide, an extract of Polymnia Sonchifolia root rich in fructo-oligosaccharide, a Lactobacillus bacteria lysate and a maltodextrin enhances the formation of the skin barrier, improving the performance of the skin barrier, promoting proper development of the epidermis and its strength, maintaining the integrity of the protein structures of the skin involved in its maintenance, in the astringent effect on the pores and filling in skin irregularities in relief, activating the ability of the skin to resist a situation of hypoxia (lack of oxygen), increasing the control exerted by the skin on the microbiota, ensuring energetic homeostasis of the skin, improving and/or reinforcing the resistance of skin to stress, and reducing inflammation.

### DISCLOSURE OF THE INVENTION

A first object of the invention concerns a cosmetic composition comprising, in a physiologically-acceptable medium, at least:

a) a cosmetic agent consisting of galactomannans of molar masses comprised between 5 and 630 kDa, preferably between 5 and 120 kDa, still more preferably between 8 and 80 kDa, and crosslinked sulfated galactans of molar masses comprised between 7 and 3000 kDa, preferably between 7 and 1100 kDa, still more preferably between 8 and 200 kDa, and
b) a prebiotic and a probiotic.

The invention also relates to a cosmetic method for care and/or makeup for keratin materials, in particular skin, lips and/or eyes, comprising at least the application onto said keratin materials of a cosmetic care and/or makeup composition, in particular a makeup composition for the complexion, lips or eyes, preferably for the complexion.

The cosmetic method is particularly intended to maintain the skin ecosystem and/or maintain the balance of the skin microbiota, in particular microbial diversity, strengthen the skin barrier and its hydrolipidic film, improve the integrity and the resistance of the skin to stresses, in particular to promote its adaptive capacities to the conditions of hypoxia and/or limit the skin consequences of a hypoxic environment, promote desquamation and/or epidermal renewal, reduce loss of firmness, reduce loss of pore elasticity, reduce surface irregularities, improve the uniformity of the complexion, promote the energetic homeostasis of the skin, and/or promote and/or improve makeup wear and the uniformity of its deposit.

The invention also concerns the non-therapeutic cosmetic use of at least one cosmetic agent consisting of glucomannans with molar masses comprised between 5 and 630 kDa and crosslinked sulfated galactans of molar masses comprised between 7 and 3000 kDa, a prebiotic and a probiotic fraction such as defined according to the invention, as a combination intended to maintain the skin ecosystem and/or maintain the balance of the skin microbiota, in particular microbial diversity, strengthen the skin barrier and its hydrolipidic film, improve the integrity and the resistance of the skin to stresses, in particular to promote its adaptive capacities to the conditions of hypoxia and/or limit the skin consequences of a hypoxic environment, promote desquamation and/or epidermal renewal, reduce loss of firmness, reduce loss of pore elasticity, reduce surface irregularities, improve the uniformity of the complexion, promote the energetic homeostasis of the skin, and/or promote and/or improve makeup wear and the uniformity of its deposit.

Definitions

According to the invention, “keratin materials” means the skin, mucosa (in particular the lips) and integuments. According to a particular embodiment, the composition is intended for topical application on the skin, lips or integuments, preferably the skin.

“Skin” means particularly the skin of the face and/or neck, in particular the skin of the face, including the more specific areas of the eyelids (included in the category of eye makeup) and the eye contour.

“Skin microbiota” or “microflora” means the microorganisms (bacteria, yeast, fungi, viruses, etc.)

present on the surface of the keratin materials and especially skin.

The term “skin microbiome” designates all these microorganisms, their genome and their interactions with their environment.

According to the invention, “skin ecosystem”, also called “skin biosphere”, means the skin microbiota and its host, the skin.

“Imbalance of the skin microbiota” means, in particular, an imbalance of the composition and/or diversity of the skin microbiota.

According to the invention, to “maintain the skin ecosystem and/or maintain the balance of the skin microbiota, in particular microbial diversity” means the ability of the active ingredients or a composition containing them to preserve or maintain the natural balance of the skin ecosystem, including under conditions of disturbance related to internal and/or external factors. To “maintain the balance of the skin microbiota” means, in particular, preserving or maintaining the composition and/or diversity of the microbiota, and controlling its development.

A “uniform complexion” means a luminous and uniform complexion, also called radiance of the complexion, as opposed to an “alteration of the complexion” which designates in particular a dull, uneven, irregular complexion, with surface and/or color irregularities. In the case of oily skin, the damage to the complexion particularly comprises a shiny complexion and a perception of discomfort and perceived manifestations such as skin imperfections or esthetic disorders.

“Surface irregularities” also called “skin imperfections”, particularly mean, according to the invention, irregularities in relief (e.g.: pores, flakes, fine lines and wrinkles, an imperfect skin texture) and/or color irregularities. For lips, these irregularities in relief may comprise chapped or cracked lips.

According to the invention, to “prevent and/or limit the skin consequences of a hypoxic environment” means, in particular, to maintain the diversity of the microbiota, and/or prevent and/or improve skin alteration related to a transient condition of hypoxia, such as a decrease in cell cohesion, a decrease in water supply to the epidermis, an increase in the production of reactive oxygen species, an inhibition of the formation of keratins for differentiation and/or an increase in collagen breakdown.

According to the invention, to “promote the adaptive capacities of the skin to conditions of hypoxia”, means the effect of the cosmetic composition of the invention on the capacity of the skin to resist a situation of hypoxia (lack of oxygen) by stimulating the expression of genes induced when the oxygen content of the skin is low and by allowing the metabolism to adapt to this new environment.

According to the invention, to “promote the energetic homeostasis of the skin” means the capacity of the cosmetic composition to ensure the energetic homeostasis of the skin by contributing to the formation of ATP, a molecule for storing cellular energy used to ensure the overall metabolism of skin cells.

“Oily skin or skin with an oily tendency” means, in particular, oily skin characterized by the excessive presence of sebum on its surface, produced by secretion of the sebaceus glands. This sebum production is more particularly ensured by sebocytes via a process of cellular differentiation and synthesis or accumulation of lipids, called lipogenesis. It is also called hyperseborrheic skin.

Oily skin is often associated with deficient desquamation, an inflammatory background and/or an oxidation of lipids, which can cause a shiny complexion, an imperfect skin texture, a perception of discomfort and manifestations felt as skin imperfections or esthetic disorders; such skin also exhibits poorer makeup wear during the day and particularly in the afternoon when sebaceous production is at a maximum.

In addition to its unsightly appearance, oily skin can be a favorable terrain for the proliferation of microorganisms and therefore participate in the occurrence of skin disorders (e.g. acne lesions). This is known as oily or hyperseborrheic skin with an acneic tendency.

“Esthetic skin disorders or nonpathological disorders associated with oily skin or skin with an oily tendency” means, in particular, disorders due to hyperseborrhea, deficient desquamation, a hypoxic environment and/or an imbalance of the microbiota. In particular, esthetic skin disorders are nonpathological disorders, selected from skin presenting an imbalance of the skin microbiota, deficient desquamation, lipid oxidation, an alteration of the complexion, and in particular a shiny complexion, a perception of discomfort, surface irregularities, in particular dilated follicular orifices or pores, an imperfect skin texture, and/or a skin exhibiting poorer makeup wear.

### DETAILED DESCRIPTION OF THE INVENTION

The invention therefore especially concerns a cosmetic composition comprising, in a physiologically-acceptable medium, at least:
a) a cosmetic agent consisting of galactomannans of molar masses comprised between 5 and 630 kDa, preferably between 5 and 120 kDa, still more preferably between 8 and 80 kDa, and crosslinked sulfated galactans of molar masses comprised between 7 and 3000 kDa, preferably between 7 and 1100 kDa, still more preferably between 8 and 200 kDa, and
b) a prebiotic and a probiotic.

### Galactomannans and Crosslinked Sulfated Galactans

The cosmetic agent within the meaning of the invention consists of biopolymers, i.e., polymers derived from plant matter, which are obtained by chemical synthesis. These biopolymers have a film-forming effect, i.e., an effect that can create a film on the skin surface that is not visible to the naked eye, in order to protect the skin from external aggressions such as pollution and allergens.

Such biopolymers are particularly described in patent applications WO2017/19792 and WO2017/129780 of the Societe Industrielle Limousine d′Application Biologique.

The cosmetic or dermocosmetic agent used according to the invention is made up of:
galactomannans of molar mass comprised between 5 and 630 kDa, and
crosslinked sulfated galactans with molar masses comprised between 7 and 3000 kDa.

“Mean molar mass” of a mixture of molecules means the weighted mean molar masses of each molecule of the mixture.

“Crosslinked”, within the meaning of the invention, means a biopolymer in which a three-dimensional network is formed by means of the formation of chemical or physical bonds between the molecules of the biopolymer.

Preferentially, the cosmetic or dermocosmetic agent consists of:
galactomannans of mean molar mass comprised between 5 and 120 kDa, and crosslinked sulfated galactans of mean molar mass comprised between 7 and 1100 kDa.

Even more preferentially, the cosmetic or dermocosmetic agent consists of:
galactomannans of mean molar mass comprised between 8 and 80 kDa, and crosslinked sulfated galactans of mean molar mass comprised between 8 and 200 kDa.

The galactomannans can be obtained in particular from the hydrolysis of native galactomannans from tara (Caesalpinia spinosa), guar (Cyamopsis tetragonoloba), locust bean (Ceratonia siliqua), as well as senna (Cassia angustifolia), cassia (Cassia fistula, Cassia obtusifolia or Cassia tora), Chinese honey locust (Gleditsia sinensis), honey locust (Gleditsia triacanthos), Chinese scholar tree (Sophora japonica) and/or fenugreek (Trigonella foenum-graecum), preferably Caesalpinia spinosa.

The crosslinked sulfated galactans can be obtained by hydrolysis of native galactans from carrageenans (Kappaphycus alvarezii, Kappaphycus striatum, Eucheuma cottonii, Eucheuma spinosum, Chondrus crispus, Gigartina skottsbergii, Sacrothalia crispata), or Furcellaria fastigiata, agar (Gelidium sesquipedale) or algae (Polysiphonia lanosa or Codium fragile), preferably Kappaphycus alvarezii.

According to one particular embodiment of the invention, the cosmetic agent is made up of galactomannans obtained by hydrolysis of native galactomannans from tara (Caesalpinia spinosa), guar (Cyamopsis tetragonoloba), locust bean (Ceratonia siliqua), as well as senna (Cassia angustifolia), cassia (Cassia fistula, Cassia obtusifolia or Cassia tora), Chinese honey locust (Gleditsia sinensis), honey locust (Gleditsia triacanthos), Chinese scholar tree (Sophora japonica) and/or fenugreek (Trigonella foenum-graecum), preferably Caesalpinia spinosa; and crosslinked sulfated galactans obtained by hydrolysis of native galactans from carrageenans (Kappaphycus alvarezii, Kappaphycus striatum, Eucheuma cottonii, Eucheuma spinosum, Chondrus crispus, Gigartina skottsbergii, Sacrothalia crispata), or Furcellaria fastigiata, agar (Gelidium sesquipedale) or algae (Polysiphonia lanosa or Codium fragile), preferably Kappaphycus alvarezii.

The galactomannans and crosslinked sulfated galactans are obtained according to the following steps:
Solubilization of the native galactomannan powder or, respectively, the native sulfated galactans in water,
Chemical or enzymatic hydrolysis;
Separation of the soluble or insoluble phases, in order to eliminate the insoluble phase;
Selection by membrane filtration(s) of galactomannans of defined molar mass.

The crosslinked sulfated galactans are also crosslinked by a crosslinking agent, preferentially an ionic crosslinking agent.

The galactomannans and crosslinked sulfated galactans thus obtained are mixed to constitute a cosmetic agent.

According to a particular and preferred embodiment, cosmetic agent a) according to the invention consists of 60 to 90% of galactomannans and 10 to 40% of crosslinked sulfated galactans.

According to a particular and preferred embodiment, the cosmetic agent consists of galactomannans obtained from Caesalpinia spinosa of molar masses comprised between 1 and 150 kDa, and crosslinked sulfated galactans obtained from Kappaphycus alvarezii of molar masses comprised between 1 and 150 kDa, in particular.

According to one particular embodiment of the invention, cosmetic agent a) is present in the composition in an amount ranging from 0.01% to 2% by weight of active ingredient relative to the total weight of the composition, preferentially 0.1% to 1%, and more preferentially still from 0.4% to 0.8% by weight of active ingredient relative to the total weight of the composition.

“Active ingredient” means the active compounds of the cosmetic agent to which the efficacy is attributed. We also talk about dry matter for plant extracts.

Such biopolymers are described in patent applications WO2017/19792 and WO2017/129780 of the Société Industrielle Limousine d'Application Biologique, in particular Example 1 of application WO2017/129780. The use of these biopolymers improves the skin barrier effect by a protective effect for the skin from the penetration of toxic molecules, such as pollutants, allergens and heavy metals. The film effect after application on the skin also improves the overall appearance of the face and promotes the wear of pigments and makeup.

According to a particular and preferred embodiment, as a cosmetic agent made up of galactomannans and crosslinked sulfated galactans, the following are used: the product sold by SILAB under the tradename FILMEXEL®, INCI name “CAESALPINIA SPINOSA EXTRACT and KAPPAPHYCUS ALVAREZII EXTRACT and WATER”, in the powder form and the composition CAESALPINIA SPINOSA EXTRACT 76%, KAPPAPHYCUS ALVAREZII EXTRACT 19%, and WATER 5% (95% of active ingredient or dry matter).

### Prebiotic/Probiotic Complex (Synbiotic)

“Prebiotic” means non-living food constituents that have the property of modulating the growth and activity of probiotic strains (lactobacilli, bifidobacteria, etc.) and other bacterial strains of the intestine beneficial to human health. Prebiotics are generally monosaccharides, disaccharides or oligosaccharides, which can be naturally found in fruits, vegetables and honey and are extractable. Others are industrially produced by hydrolysis of polysaccharides (for example fructo-oligosaccharides (FOS)) or synthesized by subjecting disaccharides such as lactose to the action of enzymes like lactases with transferase activities to produces trans-galacto-oligosaccharides (TOS) or by a chemical isomerization reaction which gives lactulose. Currently, trans-galacto-oligosaccharides (GOS) and inulin-type fructans are the ones whose prebiotic effects are recognized.

“Probiotic or derivative” means living or inactivated microorganisms which, when they are ingested in sufficient quantity, exert positive effects on health beyond traditional nutritional effects.

Probiotics can be bacteria or yeast.

Probiotics or derivatives can be chosen from among a strain of one or more of the following: Lactobacillus, strains and derivatives of Clostridia, strains and derivatives of Bifidobacterium, strains and derivatives of Saccharomyces, strains and derivatives of Lactococcus, strains and derivatives of Pediococcus, strains and derivatives of Enterococcus, strains and derivatives of Escherichia, strains and derivatives of Alcaligenes, strains and derivatives of Corynebacterium, strains and derivatives of Bacillus and strains and derivatives of Propionibacterium.

Bacteria of the Lactobacillus and Bifidobacterium genera can particularly be named. Strains identified as probiotics mainly apply to the intestinal sphere, these include: Lactobacillus acidophilus, L. casei, L. gasseri, L. paracasei, L. Rhamnosus and Bifidobacteria animal, B. breve, B. longum.

Yeasts include Saccharomyces cerevisiae var. boulardii which is found naturally in lychee.

“Synbiotics” mean combinations of probiotics and prebiotics.

According to one particular embodiment, the prebiotic is chosen in the group made up of inulin and alpha-glucan oligosaccharide.

According to one particular embodiment, the probiotic is chosen in the group made up of probiotic lysates. Preferably, the probiotic lysate is of the Bacillus genus.

According to one particular and preferred embodiment, the cosmetic composition of the invention comprises Ecoskin® as the prebiotic/probiotic complex, sold by SOLABIA, which is a mixture of alpha-glucan oligosaccharide, polymnia sonchifolia root, maltodextrin, and Lactobacillus sp bacteria, INCI name:

ALPHA-GLUCAN OLIGOSACCHARIDE and POLYMNIA SONCHIFOLIA ROOT JUICE and MALTODEXTRIN and LACTOBACILLUS

The composition of Ecoskin® is: ALPHA-GLUCAN OLIGOSACCHARIDE 70%, POLYMNIA SONCHIFOLIA ROOT JUICE 19%, MALTODEXTRIN 10%, LACTOBACILLUS 1% (corresponding to 90% active ingredient).

This is a pre/probiotic complex, dried by spray drying on maltodextrin, made of: α-gluco-oligosaccharides (GOS) obtained by enzymatic synthesis from plant substrates (corn maltose, beet sucrose), 100% pure vegetable juice rich in β-fructo-oligosaccharides (FOS) obtained by cold-pressing of Polymnia sonchifolia tubers, and a probiotic bacteria Lactobacillus (L. casei, L. acidophilus), inactivated by tyndallization and lyophilized.

The prebiotic originates from α-gluco-oligosaccharides (GOS) and β-fructo-oligosaccharides from the cold-pressed extract of Polymnia sonchifolia tubers; these prebiotics stimulate the flora of the skin.

The probiotics are inactivated Lactobacillus bacteria that stimulate the β-defensins that are involved in the skin defense system. The Lactobacillus bacterial strains (Lactobacillus casei and Lactobacillus acidophilus) that are used in Ecoskin® are previously lyophilized and tyndallized, which means that their reproductive system is inactivated by heat, preventing their development in cosmetic preparations that contain them.

According to one particular embodiment, the prebiotic/probiotic complex is present in the composition in a content ranging from 0.05% to 3% by weight of active ingredient relative to the total weight of the composition, preferentially 0.1% to 1% by weight of active ingredient relative to the total weight of the composition.

10 According to a particular embodiment, the cosmetic composition may also comprise at least one hydrolysate of Theobroma cacao L beans

### Hydrolysate of Theobroma Cacao L

The Theobroma cacao L. hydrolysate according to the invention is a peptide and sugar hydrolysate of Theobroma cacao L beans predominantly comprising peptides and sugars. “Predominantly peptides and sugars” means a quantity greater than 50%, preferentially greater than 60%, more preferentially still greater than 70% and able to reach 90% (weight/weight) of dry matter in peptides and sugars, preferentially 90% by weight of dry matter. “Peptide and sugar hydrolysate” means a hydrolysate predominantly or essentially comprising peptides and sugars (mono and oligosaccharides). The proteins and polysaccharides naturally present in the beans were hydrolyzed into peptides and into oligosaccharides and monosaccharides; advantageously the hydrolysis is an enzymatic hydrolysis.

This hydrolysate can be obtained as follows:
a) crushed Theobroma cacao L beans are dispersed in aqueous phase;
b) an enzymatic treatment of the aqueous dispersion obtained in step a) is performed;
c) the enzymatic hydrolysate is recovered by solid/liquid separation,
d) the hydrolysate is purified by ultrafiltration and nanofiltration, then possibly e) the hydrolysate obtained in step d) is lyophilized.

The hydrolysate according to the invention is obtained from Theobroma cacao L beans, as the starting material, which may include either the bean alone or the bean and its shell, preferentially beans are used including the bean and its shell. The hydrolysate of non-lyophilized cacao used according to the invention comprises from 20 to 70% of peptides and 5 to 40% of sugars. The hydrolysate of cacao lyophilized with no drying substrate according to the invention comprises more than 90% of dry matter comprising from 20 to 70% of peptides and 5 to 40% of sugars.

In another very preferred embodiment according to the invention, the peptides and sugars present in the hydrolysate used according to the invention have a molecular weight comprised between 200 Da and 10 kDa.

Thus, according to one particular and preferred embodiment, Theobroma cacao L bean hydrolysate is a peptide and sugar hydrolysate of Theobroma cacao L beans, particularly comprising peptides and sugars having a molecular weight comprised between 200 Da and 10 kDa.

The hydrolysate thus obtained can then be further diluted in water or in any mixture of solvents containing water. Thus, the cacao hydrolysate according to the invention can advantageously be diluted in one or more physiologically-acceptable solvents, such as water, glycerol, ethanol, propanediol, butylene glycol, dipropylene glycol, ethoxylated or propoxylated glycols, cyclic polyols or any mixture of these solvents.

To prepare the composition, the cacao lysate according to the invention may be in the liquid or lyophilized form. According to one particular embodiment, it is in the liquid form.

According to one particular and preferred embodiment, as Theobroma cacao L. hydrolysate, a product sold by ASHLAND under the tradename BLUMILIGHT™, INCI name “BUTYLENE GLYCOL and WATER and THEOBROMA CACAO (COCOA) SEED EXTRACT”, in the form of a liquid and the composition of BUTYLENE GLYCOL 55%, WATER 43.75%, THEOBROMA CACAO (COCOA) SEED EXTRACT 1.25% are used (1.25% of active material or dry matter).

According to one particular embodiment, the Theobroma cacao L. hydrolysate is present in the composition in a content ranging from 0.0005% to 0.025% by weight of active ingredient relative to the total weight of the composition, preferentially 0.001 to 0.01% by weight of active ingredient relative to the total weight of the composition.

### Galenic

The compositions according to the invention are intended more particularly for topical application on keratin materials, in particular on the skin.

The compositions of the invention comprise a cosmetically-acceptable medium, i.e., compatible with skin and integuments. The compositions may have any cosmetic form, and especially be in the form of creams, oil-in-water or water-in-oil emulsions or multiple emulsions, solutions, suspensions, gels, milks, lotions, serums, sticks or powders, and suited to application on the skin, lips and/or integuments.

According to one particular embodiment, the composition of the invention is intended for topical application on the skin, lips or eyes and is present in the form of a care product and/or a makeup product for the skin, lips and/or eyes, in particular for the complexion.

We will use the terms skin makeup and complexion makeup interchangeably.

“Eye makeup” means, in particular, eyeliners or eyeshadows.

According to one particular and preferred embodiment, the composition of the invention is present in the form of a foundation or a primer, preferably a foundation.

According to one particular and preferred embodiment, the composition of the invention is present in the form of a powder foundation, a foundation or a primer, preferably a foundation.

The cosmetic composition according to the invention is advantageously in the form of an oil-in-water emulsion, a water-in-oil emulsion, a multiple emulsion or an aqueous gel.

The composition is preferably in the form of an emulsion containing an aqueous phase and an oily phase.

The aqueous phase generally represents from 1 to 99% by weight with regard to the total weight of said composition,

The composition of the invention also comprises an oily phase in addition to the aqueous phase. An oily phase according to the invention can comprise hydrocarbon oils, silicone oils, fluorinated or nonfluorinated, and mixtures thereof. These oils can be volatile or nonvolatile, vegetable, mineral or synthetic.

Advantageously, hydrocarbon oils are used.

Volatile hydrocarbon oils include C8-C16 branched alkanes, C8-C16 branched esters and mixtures thereof.

volatile hydrocarbon oils include, in particular, hydrocarbon oils, vegetable hydrocarbon oils, C10-C40 synthetic ethers, C10-C40 synthetic esters, C12-C26 fatty alcohols, C12-C22 higher fatty acids, and mixtures thereof.

The oils can be present in the composition of the invention in a content ranging from 1 to 95% by weight relative to the total weight of the composition.

The composition of the invention can also comprise any additive usually used in cosmetics, such as UV filters, antioxidants, surfactants, gelling agents, fillers, coloring materials, preservatives, film-forming polymers, fragrances, cosmetic active ingredients, such as, for example, emollients, moisturizers, vitamins, anti-aging agents, lightening agents and mixtures thereof.

According to one particular embodiment of the invention, the cosmetic composition comprises at least one or more coloring materials.

The coloring materials may be chosen from water-soluble or non-water soluble, fat-soluble or non-fat-soluble, organic or inorganic coloring materials, materials with optical effects and mixtures thereof.

Within the meaning of the present invention, coloring material means a compound that can produce a colored optical effect when it is formulated in sufficient quantity in an appropriate cosmetic medium.

According to one particular embodiment, the coloring material(s) are notably chosen from mineral and/or organic pigments, composite pigments (based on mineral and/or organic materials), colorants, nacres or pearlescent pigments, and mixtures thereof.

Pigments mean white or colored particles, inorganic (mineral) or organic, insoluble in the liquid organic phase, intended to color and/or opacify the composition and/or the deposit made with the composition.

Mineral pigments include, for example, titanium dioxide (rutile or anatase), optionally surface treated; black, yellow, red and brown iron oxides; manganese violet; ultramarine blue; chromium oxide; hydrated chromium oxide and Prussian blue.

For compositions intended for the lips, pigments include titanium dioxide, black, red and brown iron oxide and manganese violet.

Organic pigments include, for example D & C Red 19; D & C Red 9; D & C Red 22; D & C Red 21; D & C Red 28; D & C Yellow 6; D & C Orange 4; D & C Orange 5; D & C Red 27; D & C Red 13; D & C Red 7; D & C Red 6; D & C Yellow 5; D & C Red 36; D & C Red 33; D & C Orange 10; D & C Yellow 6; D & C Red 30; D &C Red 3; D &C Blue 1; carbon black and cochineal carmine lacquers.

Water soluble colorants include Yellow 5, Yellow 6, Blue 1, Green 5, Green 3, Green 6, Orange 4, Red 4, Red 21, Red 22, Red 27, Red 28, Red 33, Red 40 and cochineal carmine (CI 15850, CI 75470).

Fat-soluble colorants are, for example Sudan red, D&C Red 17, D&C Green 6, beta-carotene, soy oil, Sudan brown, D&C Yellow 11, D&C Violet 2, D&C Orange 5, quinoline yellow and annatto.

Nacres or pearlescent pigments can be chosen from white pearlescent pigments, such as mica coated with titanium oxide, bismuth oxychloride; and colored pearlescent pigments, such as titanium mica with iron oxides, titanium mica with Prussian blue or chromium oxide, titanium mica with an organic pigment of the aforementioned type, as well as pigments based on bismuth oxychloride. Ranges of pearlizers of commercial reference Reflecks®, Ronastar®, Timiron® and Syncrystal® can be named.

In particular, the coloring materials are present in the composition in a content ranging from 2% to 30% by weight, preferably from 4% to 15% by weight, relative to the total weight of the composition.

According to one particular embodiment, the composition of the invention comprises pigments, in particular mineral pigments in a content ranging from 5 to 25%, notably from 10 to 20% by weight relative to the total weight of the composition.

Fillers are chosen, in particular, from silicas, micas, of natural or synthetic origin, kaolin, zinc and titanium oxides; calcium carbonate, magnesium carbonate and bicarbonate; zinc, magnesium or lithium stearate, zinc laurate, magnesium myristate; powders of synthetic polymers, such as polyethylene, polyesters, polyamides (for example nylon); powders of polyacrylic acid or poly(methacrylic acid), powders of silicone resin; mineral powders such as spherical silica; spherical titanium dioxides; glass and ceramic beads; powders of organic materials of natural origin such as starches from corn, wheat, rice, crosslinked or non-crosslinked, and mixtures thereof.

According to one particular embodiment, the composition of the invention comprises pigments, in particular mineral pigments in a content ranging from 5 to 25%, notably from 10 to 20% by weight relative to the total weight of the composition.

According to one particular embodiment of the invention, the cosmetic composition does not contain cosmetic active ingredients other than those that are the subject of the invention.

According to another particular embodiment of the invention, the composition also contains other cosmetic and/or dermatological active ingredients. Active ingredients particularly include those seeking to stimulate biodiversity and/or homeostasis of the skin microbiota, cellular renewal, regeneration or revitalization of the complexion (radiance), reduction of sebum production and protection from external aggressions.

The following active agents can be mentioned, in particular, intended to stimulate:
biodiversity and homeostasis of the skin microbiota, like the active ingredient Actibiome© (Water and seawater and glycerin and Laminaria digitata extract and Chlorella vulgaris extract and saccharide isomerate and phenoxyethanol and ethylhexylglycerin), by rebalancing the pH of skin previously decreased by a period of stress;
cellular renewal, by means of peeling active ingredients alone or in combination and at acidic pH.
   chemically, such as alpha hydroxy acids (AHA) (glycolic, lactic, citric, etc. acid), beta hydroxy acids (BHA) (salicylic acid), polyhydroxy acids (PHA) (gluconolactone);
   biologically, such as Exfolactive C EL PX© (Opuntia coccinellifera), DERMOCH DP© (Chlorella vulgaris), ALGOWHITE© (Ascophyllum nodosum)
   enzymatically, such as MELACLEAR© (Gluconic acid, Sutilains);
the regeneration or revitalization of the complexion (radiance) using the following types of active ingredients:
   smoothing, such as retinol (vitamin A-like)
   lightening by acting on melanogenesis (vitamin C derivative)
   energizing, such as nutrients and/or vitamin C and E antioxidants, such as SEPIVITAL© (potassium ascorbyl tocopheryl phosphate)
reduction of sebum production, using sebum captors (clays) or sebum regulating active ingredients such as zinc gluconate (Mineralis GU/Zn) and lipophilic avocado extract (5 alpha avocuta),
protection from external aggressions: pollution, blue light, UV, using chemical or mineral filters and/or diffusing reflective particles, and mixtures thereof.

The invention also relates to a cosmetic method for care and/or makeup for keratin materials, in particular skin, lips and/or eyes, comprising at least the application onto said keratin materials of a cosmetic care and/or makeup composition as defined according to the invention, in particular a makeup composition for the complexion, lips or eyes, preferably for the complexion. The cosmetic method is particularly intended to maintain the skin ecosystem and/or maintain the balance of the skin microbiota, in particular microbial diversity, strengthen the skin barrier and its hydrolipidic film, improve the integrity and the resistance of the skin to stresses, in particular to promote its adaptive capacities to the conditions of hypoxia and/or limit the skin consequences of a hypoxic environment, promote desquamation and/or epidermal renewal, reduce loss of firmness, reduce loss of pore elasticity, reduce surface irregularities, improve the uniformity of the complexion, promote the energetic homeostasis of the skin, and/or promote and/or improve makeup wear and the uniformity of its deposit.

The invention also concerns the use of a nontherapeutic cosmetic product of at least one cosmetic agent consisting of glucomannans of molar masses comprised between 5 and 630 kDa and crosslinked sulfated galactans of molar masses comprised between 7 and 3000 kDa, a prebiotic and a probiotic fraction such as defined according to the invention, as a combination intended to maintain the skin ecosystem and/or maintain the balance of the skin microbiota, in particular microbial diversity, strengthen the skin barrier and its hydrolipidic film, improve the integrity and the resistance of the skin to stresses, in particular to promote its adaptive capacities to the conditions of hypoxia and/or limit the skin consequences of a hypoxic environment, promote desquamation and/or epidermal renewal, reduce loss of firmness, reduce loss of pore elasticity, reduce surface irregularities, improve the uniformity of the complexion, promote the energetic homeostasis of the skin, and/or promote and/or improve makeup wear and the uniformity of its deposit.

The invention will be illustrated by the following nonlimiting examples. The percentages are expressed as percentage by weight relative to the total weight of the composition unless otherwise indicated.

### EXAMPLES

### Example 1

### Effect of the Combination of a Caesalpinia Spinosa Extract and a Kappaphycus Alvarezii Extract (Filmexel®) in Combination with Ecoskin® on Keratinocytes

A study on the effects on transcription of these active ingredients, used alone or in combination, was performed using TaqMan low density array (TLDA). Via this technology, the modulation of the expression of genes coding for specific proteins is studied in response to a treatment of normal human keratinocytes, which allows mapping the effect on gene expression of active ingredients alone or in combination.

In particular, this approach shows the beneficial effects of active ingredients or combinations thereof in the fields covered by the profile: formation of the epidermis and desquamation, extracellular matrix, detoxification and antioxidation, inflammation and innate immunity.

### Materials and Methods

Cells used: Normal human epidermis keratinocytes

Culture medium: EpiLife® supplemented by HKGS (Human Keratinocyte Growth Supplement)

Cutaneous origin of the cells: abdominal sample, 47-year old woman

Study model: TaqMan low density array (TLDA)

Treatment of cells by the compounds: 24 h

Compounds tested:
ECOSKIN® (ALPHA-GLUCAN OLIGOSACCHARIDE and POLYMNIA SONCHIFOLIA ROOT JUICE and MALTODEXTRIN and LACTOBACILLUS) at 0.25%
FILMEXEL® (CAESALPINIA SPINOSA EXTRACT and KAPPAPHYCUS ALVAREZII EXTRACT and WATER) at 0.25%
ECOSKIN® a 0.25% +FI LMEXEL® at 0.25%

### Results

Table 1 below reports the activity variations for the genes studied.

The experimental conditions used are the following:
Each test is done in triplicate (3 untreated and 3 treated under the same conditions);
Fisher's F test is first applied by comparing the two data matrices. When the value is greater than α=0.05 then the variance of the Student's t-test is 2, when the Fisher's F-test is less than α=0.05, then the variance will be equal to 3.
The transcriptional variations used (significant) will be those that correspond to a Student's t-test of less than or equal to α (or p)=0.05. The stimulations are in dark gray (see below); the white background indicates nonsignificant variations, i.e., those corresponding to a Student's t-test greater than alpha (or p)=0.05.

TABLE 1

| | | | | | Filmexcel ® |
|---|---|---|---|---|---|
| | | | | | 0.25% + |
| | | | Ecoskin ® | Filmexcel ® | Ecoskin ® |
| Gene functions | Gene | Symbol | 0.25% | 0.25% | 0.25% |
| Constituent | Involucrin | IVL | 1.311 | 1.284 | 1.803 |
| protein of the | | | | | |
| corneocyte envelope | | | | | |
| (barrier formation) | | | | | |
| Protease | Serpin | SERPINB7 | 1.075 | 1.508 | 1.843 |
| inhibition | peptidase | | | | |
| (integrity of | inhibitor, | | | | |
| the epidermis | Clade B | | | | |
| and its barrier) | (Ovalbumin), | | | | |
| | Member 7 | | | | |
| Protease | Serpin | SERPINB3 | 1.936 | 2.562 | 4.208 |
| inhibition | peptidase | | | | |
| (integrity of | inhibitor, | | | | |
| the epidermis | Clade B, | | | | |
| and its barrier) | Member 3 | | | | |
| Formation of ATP, | ATPase, Ca++ | ATP2C1 | 1.042 | 1.073 | 1.161 |
| energy storage | transporting, | | | | |
| molecules | type 2C, | | | | |
| (cellular energy) | member 1 | | | | |
| Regenerative | Keratin 5 | KRT5 | 1.094 | 1.177 | 1.291 |
| keratinocyte | | | | | |
| cytoskeleton | | | | | |
| (mechanical | | | | | |
| strength of | | | | | |
| the epidermis) | | | | | |
| Formation of tight | Occludin | OCLN | 1.163 | 1.237 | 1.444 |
| junctions (quality | | | | | |
| of the epidermal | | | | | |
| barrier) | | | | | |
| Formation of tight | Claudin-1 | CLDN1 | 1.831 | 1.705 | 2.185 |
| junctions (quality | | | | | |
| of the epidermal | | | | | |
| barrier) | | | | | |
| Lipid transport | Solute carrier | SLC27A4 | 1.022 | 1.165 | 1.230 |
| (formation of | family 27 | | | | |
| the lipid | (fatty acid | | | | |
| barrier) | transporter), | | | | |
| | member 4 | | | | |
| Hypoxia adaptation | Hypoxia | HIF1a | 0.995 | 1.178 | 1.257 |
| factor (cellular | inducible | | | | |
| resistance to lack of | factor 1 | | | | |
| oxygen) | alpha | | | | |
| Antimicrobial peptide, | Elafin | PI3 | 1.380 | 0.88 | 1.943 |
| constituent of the | (SKALP) | | | | |
| corneocyte envelope, | | | | | |
| anti-inflammatory with | | | | | |
| anti-protease activity | | | | | |
| (control of flora | | | | | |
| development, barrier | | | | | |
| strength, soothing and | | | | | |
| maintaining integrity | | | | | |
| of the barrier) | | | | | |
| Control of epidermal | Suppressor | SOCS3 | 0.830 | 1.1 | 1.348 |
| differentiation | of cytokine | | | | |
| (epidermal formation) | signaling 3 | | | | |
| Control of epidermal | Transforming | TGFBR1 | 1.198 | 1.262 | 1.524 |
| differentiation (skin | growth factor | | | | |
| surface formation) | beta receptor 1 | | | | |
| Elimination of toxic | Glutathione S | GSTZ1 | 0.875 | 1.162 | 1.313 |
| molecules by | transferase | | | | |
| conjugation with | zeta 1 | | | | |
| glutathione | | | | | |
| (detoxification) | | | | | |

These data show a synergistic effect for the combinations tested, allowing:
enhancing the formation of the skin barrier, in particular the formation of corneocytes (cellular envelope) by stimulating the transcriptional activity of IVL and PI3 genes.
improving the performance of the skin barrier by stimulating the transcriptional activity of CLDN1 and OCLN genes, involved in the formation of intercellular tight junctions that limit the passage of intercellular water (insensible water loss) and maintain the epidermal lipids at the skin surface, and by stimulating the transcriptional activity of SLC27A4 gene involved in epidermal lipid transport
promoting the proper development of the epidermis and its strength, in particular by stimulating the transcriptional activity of SOCS3, TGFBR1 and KRT5 genes,
maintaining the integrity of the protein structures of the skin involved in its maintenance, in the astringent effect on the pores and on filling skin irregularities in relief, preventing the degradation of these protein structures by activation of protease inhibitor genes such as SERPINB3, SERPINB7 and PI3
activating the ability of skin to resist a situation of hypoxia (lack of oxygen) stimulating the transcriptional activity of HIF1a induced when the oxygen content of the skin is low and permits the metabolism to adjust to this new tissue environment.
increasing the control exerted by the skin on the microbiota by stimulating the 20 transcriptional activity of PI3 gene, antimicrobial bactericidal peptide.
ensuring the energetic homeostasis of the skin by stimulating the transcriptional activity of gene ATP2C1 which contributes to the formation of ATP, molecule for storing cellular energy used to ensure the overall metabolism of skin cells.
improving and/or strengthening the resistance of the skin to stress by protecting it from toxins by stimulation of the transcriptional activity of GSTZ1, which eliminates them by coupling with the glutathione molecule, while limiting inflammation by stimulating the transcriptional activity of PI3 gene.

These results show that the cosmetic composition of the invention helps to maintain the skin ecosystem and/or maintain the balance of the skin microbiota, in particular microbial diversity, strengthen the skin barrier and its hydrolipid film, improve the integrity and strength of the skin, promote its ability to adapt to conditions of hypoxia, enhance detoxification processes and modulate inflammation, reduce loss of firmness, reduce loss of pore elasticity and the formation of imperfections in skin relief (fine lines and wrinkles), promote and/or improve the uniformity of the distribution of a makeup product, and provide the skin with the necessary energy to improve the radiance of tired and dull skin.

### Example 2

### Cosmetic Formulations

The cosmetic formulations are prepared according to the conventional formulation methods. The percentages are expressed in weight percent of ingredient (raw material) relative to the total weight of the composition.

### A Moisturizing Day Cream for Combination Skin:

| VEGETABLE GLYCEROL | |
|---|---|
| (Glycerine) | 6.0% |
| FILMEXEL ® | |
| (Caesalpinia spinosa fruit extract and | 0.50% |
| Kappaphycus alvarezii extract and water) | |
| ECOSKIN ® | |
| (Alpha-glucan oligosaccharide and Polymnia | |
| sonchifolia root juice and Maltodextrin | 0.50% |
| and Lactobacillus) | |
| RENOHYAL | |
| (Sodium hyaluronate) | 0.10% |
| Isononyl isononanoate | 10.0% |
| Steareth-2 | 12.5% |
| Glyceryl stearate | 1.1% |
| Stearyl alcohol | 5.0% |
| Butylene glycol | 3.0% |
| Glycerine | 2.0% |
| Preservative | 0.5% |
| Water, qs | 100% |

### A Gel for Combination Skin:

| VEGETABLE GLYCEROL | |
|---|---|
| (Glycerine) | 6.0% |
| ECOSKIN ® | |
| (Alpha-glucan oligosaccharide and Polymnia | |
| sonchifolia root juice and Maltodextrin | 0.50% |
| and Lactobacillus) | |
| FILMEXEL ® | |
| (Caesalpinia spinosa fruit extract and | 1.00% |
| Kappaphycus alvarezii extract and water) | |
| RENOHYAL | |
| (Sodium hyaluronate) | 0.1% |
| Glycol | 3.0% |
| AMPS polymer | 3.0% |
| Mineral oil | 2.0% |
| Polyethylene glycol | 1.5% |
| Preservative | 0.5% |
| Fragrance concentrate | 0.3% |
| Water, qs | 100% |

### A Foundation

| ECOSKIN ® | |
|---|---|
| (Alpha-glucan oligosaccharide and Polymnia | |
| sonchifolia root juice and Maltodextrin | 0.50% |
| and Lactobacillus) | |
| FILMEXEL ® | |
| (Caesalpinia spinosa fruit extract and | 0.50% |
| Kappaphycus alvarezii extract and water) | |
| IRON OXIDES AND TITANIUM DIOXIDE | |
| (Black CI 77499, Red CI 77491, | 18.0% |
| Yellow CI 77492, White CI 77891) | |
| Cyclopentasiloxane and cyclohexasiloxane | 5.0% |
| Cetyl dimethicone | 1.0% |
| Caprylic/capric triglycerides | 2.2% |
| Octyl stearate | 1.4% |
| Mineral oil | 3.5% |
| Beeswax | 0.8% |
| Methyl polymethacrylate | 1.1% |
| Fragrance concentrate | 0.1% |
| Water, qs | 100% |

A Foundation with SPF

| BLUMILIGHT ™ | |
|---|---|
| (Butylene glycol and Water and | 0.10% |
| Theobroma cacao (cocoa) seed extract) | |
| FILMEXEL ® | |
| (Caesalpinia spinosa fruit extract and | 0.50% |
| Kappaphycus alvarezii extract and water) | |
| ECOSKIN ® | |
| (Alpha-glucan oligosaccharide and Polymnia | |
| sonchifolia root juice and Maltodextrin | 0.50% |
| and Lactobacillus) | |
| UV-TITAN M160 | |
| (Titanium dioxide, TiO₂ (nano)) | 2.0% |
| ZINC OXIDE | |
| (Zinc oxide, CI 77947) | 3.0% |
| IRON OXIDES AND TITANIUM DIOXIDE | |
| (Black CI 77499, Red CI 77491, | 16.0% |
| Yellow CI 77492, White CI 77891) | |
| Cyclopentasiloxane and cyclohexasiloxane | 5.0% |
| Cetyl dimethicone | 1.0% |
| Caprylic/capric triglycerides | 2.2% |
| Octyl stearate | 1.4% |
| Mineral oil | 3.5% |
| Beeswax | 0.8% |
| Methyl polymethacrylate | 1.1% |
| Fragrance concentrate | 0.1% |
| Water, qs | 100% |

1.-12. (canceled)

13. Cosmetic composition comprising, in a physiologically-acceptable medium at least:
(a) a cosmetic agent consisting of galactomannans of molar masses between 5 and 630 kDa and crosslinked sulfated galactans of molar masses between 7 and 3000 kDa, and
(b) a prebiotic and a probiotic.

14. Cosmetic composition according to claim 13, wherein the cosmetic agent consists of galactomannans obtained by hydrolysis of native galactomannans from tara (Caesalpinia spinosa), guar (Cyamopsis tetragonoloba), locust bean (Ceratonia siliqua), as well as senna (Cassia angustifolia), cassia (Cassia fistula, Cassia obtusifolia or Cassia tora), Chinese honey locust (Geditsia sinensis), honey locust (Gleditsia triacanthos), Chinese scholar tree (Sophora japonica) and/or fenugreek (Trigonella foenum-graecum), preferably Caesa/pinia spinosa; and crosslinked sulfated galactans obtained by hydrolysis of native galactans from carrageenans (Kappaphycus alvarezii, Kappaphycus striatum, Eucheuma cottonii, Eucheuma spinosum, Chondrus crispus, Gigartina skottsbergii, Sacrothalia crispata), or Furcellaria fastigiata, agar (Gelidium sesquipedale) or algae (Polysiphonia lanosa or Codium fragile).

15. Cosmetic composition according to claim 13, wherein the cosmetic agent (a) consists of from 60 to 90% of galactomannans and 10 to 40% of crosslinked sulfated galactans.

16. Cosmetic composition according to claim 13, wherein the cosmetic agent consists of galactomannans obtained from Caesalpinia spinosa of molar masses comprised between 1 and 150 kDa, and crosslinked sulfated galactans obtained from Kappaphycus alvarezii of molar masses comprised between 1 and 150 kDa.

17. Cosmetic composition according to claim 13, wherein the cosmetic agent (a) is present in the composition in an amount ranging from 0.01% to 2% by weight of active ingredient relative to the total weight of the composition.

18. Cosmetic composition according to claim 13, wherein it comprises, as said prebiotic, at least one alpha-gluco-oligosaccharide, and as said probiotic, at least one Bacillus-type lysate.

19. Cosmetic composition according to claim 18, wherein the prebiotic and probiotic are in the form of a pre/probiotic complex comprising an alpha-gluco-saccharide, a Polymnia Sonchifolia root extract rich in fructo-oligo-saccharide, a Lactobacillus lysate, and a maltodextrin.

20. Cosmetic composition according to claim 13, wherein the prebiotic and probiotic is present in the composition in a total content of active ingredient ranging from 0.05% to 3% by weight of active ingredient relative to the total weight of the composition.

21. Cosmetic composition according to claim 13, wherein it is present in the form of a care and/or makeup product for the skin, lips, or eyes.

22. A cosmetic method for care and/or makeup for keratin materials, comprising at least the application onto said keratin materials of a cosmetic care and/or makeup composition comprising, in a physiologically-acceptable medium at least: (a) a cosmetic agent consisting of galactomannans of molar masses comprised between 5 and 630 kDa, and crosslinked sulfated galactans of molar masses comprised between 7 and 3000 kDa and (b) a prebiotic and a probiotic.

23. The cosmetic care and/or makeup method for the skin according to claim 22, wherein it is intended to maintain the skin ecosystem and/or maintain the balance of the skin microbiota, in particular microbial diversity, strengthen the skin barrier and its hydrolipidic film, improve the integrity and the resistance of the skin to stresses, in particular to promote its adaptive capacities to the conditions of hypoxia and/or limit the skin consequences of a hypoxic environment, promote desquamation and/or epidermal renewal, reduce the loss of firmness, reduce loss of pore elasticity, reduce surface irregularities, improve the uniformity of the complexion, promote the energetic homeostasis of the skin, and/or promote and/or improve makeup wear and the uniformity of its deposit.

24. A nontherapeutic cosmetic method to maintain the skin ecosystem and/or maintain the balance of the skin microbiota, in particular microbial diversity, strengthen the skin barrier and its hydrolipidic film, improve the integrity and the resistance of the skin to stresses, in particular to promote its adaptive capacities to the conditions of hypoxia and/or limit the skin consequences of a hypoxic environment, promote desquamation and/or epidermal renewal, reduce loss of firmness, reduce loss of pore elasticity, reduce surface irregularities, improve the uniformity of the complexion, promote the energetic homeostasis of the skin, and/or promote and/or improve makeup wear and the uniformity of its deposit comprising the application of a combination of at least one cosmetic agent consisting of glucomannans of molar masses comprised between 5 and 630 kDa and crosslinked sulfated galactans of molar masses comprised between 7 and 3000 kDa, and a prebiotic and a probiotic.
